(19) United States
(12) Patent Application Publication
(10) Pub. No.: US 2016/0361293 A1
(43) Pub. Date: Dec. 15, 2016

(54) NANOSTRUCTURED COMPOSITION COMPRISING INDOMETHACINE, ITS PHARMACEUTICALLY ACCEPTABLE SALTS AND CO-CRYSTALS AND PROCESS FOR THE PREPARATION THEREOF

(71) Applicant: DARHOLDING KFT., Budapest (HU)

(72) Inventors: Genovéva FILIPCSEI, Budapest (HU); Zsolt ÖTVÖS, Csongrád (HU); Gábor HELTOVICS, Budapest (HU); Ferenc DARVAS, Budapest (HU)

(73) Assignee: DARHOLDING KFT., Budapest (HU)

(21) Appl. No.: 15/121,673

(22) PCT Filed: Feb. 25, 2015
(86) PCT No.: PCT/HU2015/000020

§ 371 Date: Aug. 25, 2016

(30) Foreign Application Priority Data
Feb. 25, 2014 (HU) P1400104

(51) Int. Cl. A61K 31/405; A61K 9/16

(52) U.S. Cl. CPC A61K 31/405; A61K 9/1635; A61K 9/1682

(57) ABSTRACT

The invention relates to a nanostructured (nanoparticular) composition comprising Indomethacine, its pharmaceutically acceptable salts and co-crystals, processes for the preparation thereof, and compositions useful for pharmaceutical applications. The size of the nanoparticles according to the invention is smaller than 500 nm. Indomethacine (INN) or Indomethacine (USAN, previously BAN) is a non-steroidal anti-inflammatory drug (NSAID), which is used for the treatment of fever, inflammation, spasm, swells and inflammations. The machanism of action of Indomethacine is the inhibition of the synthesis of prostaglandin. It is marketed under the trade names of Indocin, Indocid, Indochron E-R, and Indocin-SR.

### FIELD OF THE INVENTION

The present invention is directed to nanostructured (nanoparticulated) compositions containing indomethacin or pharmaceutically acceptable salts or cocrystals thereof, process for the preparation thereof and pharmaceutical formulations containing them.

The nanoparticles formed from indomethacin or pharmaceutically acceptable salts or cocrystals thereof according to the invention have an average particle size of less than 500 nm.

Indometacin (INN) or indomethacin (USAN and former BAN) is a nonsteroidal anti-inflammatory drug (NSAID) used in the treatment of fever, inflammation, tense muscles, swellings and inflammation. indomethacin acts by inhibiting prostaglandin synthesis. indomethacin is marketed under the trade names Indocin, Indocid, Indochron E-R, and Indocin-SR.

### BACKGROUND OF THE INVENTION

A. Background Regarding to Nanoparticle Formation/Production

The use of nanoparticles for pharmaceutical applications requires the development of new technologies. The drugs containing nanoparticles which produced with said new technologies may have higher rate of absorption and efficiency. In addition to the efficiency improvement, the binding of the drugs to its target receptor can be controlled and such that the receptor's activity can be manipulated. Preparation of nanoparticulated drugs provides the use of controlled release systems. Auxiliary materials should be compatible, easy to bind with a particular drug, and able to degrade into fragments after use that are either metabolized or driven out via normal excretory routes.

Different approaches are known to produce the active ingredient (API) in nanoparticulate form.

Preparation method and use of API nanoparticles are described, for example, in WO/1988/001862, WO/1996/024339, WO/2008/058054, WO/2006/109177, WO/2005/048997, WO/2007/115261, U.S. Pat. No. 4,442,051, U.S. Pat. No. 4,885,279 and US 20070098802.

The API nanoparticles can be made using, for example, milling, homogenization, precipitation techniques, or supercritical fluid techniques, as is known in the art. Methods of making nanoparticulate compositions are also described in U.S. Pat. No. 5,718,388, U.S. Pat. No. 5,862,999, U.S. Pat. No. 5,665,331, U.S. Pat. No. 5,543,133, U.S. Pat. No. 5,534,270, U.S. Pat. No. 5,510,118, and U.S. Pat. No. 5,470,583.

B. Indomethacin

Indomethacin is a non-steroidal anti-inflammatory indole derivative described chemically as 1-(4-chlorobenzoyl)-5-methoxy-2-methyl-1H-indole-3-acetic acid. It is practically insoluble in water and sparingly soluble in alcohol. It has a pKa of 4.5 and is stable in neutral or slightly acidic media and decomposes in strong alkali. The suspension of indomethacin has a pH value of 4.0 to 5.0. Its structural formula is

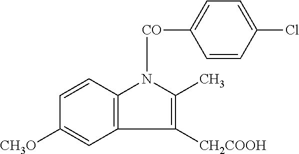

Indomethacin is white crystalline powder and its water solubility is 0.05 mg/mL.

The method of its preparation is disclosed for example in WO/1994/014784 and WO/1997/025029.

Indocin is available as capsule containing 25 mg or 50 mg of indomethacin. Indocin suspension for oral administration comprises 25 mg of indomethacin per 5 ml, indomethacin for rectal administration is provided as a rectal suppository containing 50 mg of indomethacin.

Pharmacological Properties

Following single oral doses of Indocin capsules 25 mg or 50 mg indomethacin is immediately absorbed attaining peak plasma concentration of 1-2 μg/ml at about two hours. Orally administered indomethacin capsules are virtually 100% bioavailable, with 90% of the dose absorbed within 4 hours. The effect of a single 50 mg Indocin suspension administered orally is equivalent to that of 50 mg Indocin capsule administered with food.

Indomethacin is eliminated via renal excretion, metabolism, and biliary excretion. The mean half-life of indomethacin is estimated to be about 4.5 hours. With a typical therapeutic regimen of 25 or 50 mg, the steady-state plasma concentrations of indomethacin are an average 1.4 times those following the first dose.

Absorption and Distribution

The rate of rectal absorption is greater than the rate of absorption from Indocin capsule. The amount absorbed from the suppository is equivalent to the amount absorbed from the capsule. Controlled clinical studies have shown, however, that the amount of indomethacin absorbed from suppository is less (80-90%) than the amount absorbed from Indocin capsule. The reason for this is probably that the residence time of the drug in the rectum is lower than is required for full absorption. Although it is dissolved by the rectum faster than it melts, it rarely reaches the desired effect when it is hold in the rectum for less than a few minutes by the patient.

Side Effects

Since indomethacin is a non-selective cyclooxygenase (COX) inhibitor, it inhibits both COX-1 and COX-2 which produces prostaglandins from arachidonic acid in the stomach and intestines. indomethacin, therefore, like other non-selective COX inhibitors can cause peptic ulcers. These ulcers can result in serious bleeding and/or perforation requiring hospitalization of the patient. It can cause death in some cases.

To reduce the possibility of peptic ulcers, indomethacin should be prescribed at smaller dosage, usually between 50-200 mg/day. It should always be taken with food. An ulcer protective drug (e.g. highly dosed antacids, ranitidine 150 mg at bedtime, or omeprazole 20 mg at bedtime) is recommended to all patients. Other common gastrointestinal complaints, including dyspepsia, heartburn and mild diarrhea are less serious and rarely require discontinuation of indomethacin.

Many NSAIDs, but particularly indomethacin, cause lithium retention by reducing its excretion by the kidneys. Thus indomethacin users have an elevated risk of lithium toxicity. For patients taking lithium (e.g. for treatment of depression or bipolar disorder), less toxic NSAIDs such as sulindac or aspirin, are preferred.

Indomethacin also increases plasma renin activity and aldosterone levels, and increases sodium and potassium retention. It also enhances the effects of vasopressin.

The drug may also cause elevations of serum creatinine and more serious renal damage such as acute renal failure, chronic nephritis and nephrotic syndrome. These conditions also often begin with edema and hyperkalemia.

Additionally, indomethacin quite often causes headache (10 to 20%), sometimes with vertigo and dizziness, hearing loss, tinnitus, blurred vision (with or without retinal damage), and worsens Parkinson's disease, epilepsy, and psychiatric disorders. Cases of life-threatening shock (including angioedema, sweating, severe hypotension and tachycardia as well as acute bronchospasm), severe or lethal hepatitis and severe bone marrow damage have all been reported. Skin reactions and photosensitivity are also possible side effects.

Due to its strong antipyretic activity indomethacin may obscure the clinical course of serious infections.

The frequency and severity of side effects and the availability of better tolerated alternatives make indomethacin today a drug of second choice. Its use in acute gout attacks and in dysmenorrhea is well-established because in these indications the duration of treatment is limited to a few days only, therefore serious side effects are not likely to occur.

Because of the low solubility of indomethacin in water (0.05 mg/ml) and its side effects, there is a need to develop such methods which enhance the lipophilicity/bioavailability/increase the absorption/reduce the side effect/decrease the dosage/reduce the food effect. These properties can not be improved using the formulation methods described in the art. These problems can be solved by surface modification to decrease the first pass effect or modify the metabolism of indomethacin. Beside the traditional formulation of indomethacin, the transdermal application could increase the bioavailability and decrease the time which is needed to reach the desired effect of indomethacin. The present invention satisfies this need.

### DESCRIPTION OF THE INVENTION

The present invention describes the nanostructured (nanoparticulated) composition of indomethacin or pharmaceutically acceptable salts or cocrystals thereof with enhanced lipophilicity/bioavailability/increased absorption/dissolution rate and solubility and with reduced side effect/decreased dosage.

As exemplified in the examples below, not every combination of stabilizer will result in a stable nanoparticle formation. It was discovered, that stable nanoparticles containing indomethacin can be made in flow reactor, preferably in microfluidic based flow reactor, using appropriate stabilizers.

The expression indomethacin is generally used for indomethacin and its pharmaceutically acceptable salts and cocrystals thereof.

In the nanostructured composition of the invention the nanoparticles have an average particle size of 500 nm or less. In the nanostructured composition of the invention the nanoparticles have an average particle size between 500 nm and 50 nm, preferably 300 nm and 100 nm.

Further aspect of the invention is a stable nanostructured indomethacin composition comprising:

(a) indomethacin or pharmaceutically acceptable salts or cocrystals and mixtures thereof; and

(b) at least one polyelectrolyte and/or stabilizer or a mixture thereof for stabilization of the nanoparticles, and optionally further stabilizers for steric and electrostatic stabilization;

wherein the nanoparticles have an average particle size of 500 nm or less.

The composition of the invention is prepared in a flow reactor, preferably in a microfluidic based flow reactor.

In the composition of the invention: (a) the indomethacin or pharmaceutically acceptable salts or cocrystals thereof is present in an amount selected from the group consisting of from 99.5% to 0.001%, from 95% to 0.1%, and from 90% to 0.5%, by weight, based on the total combined weight of the indomethacin or pharmaceutically acceptable salts or cocrystals thereof and at least one stabilizer or polyelectrolyte, including other excipients; (b) the stabilizer or polyelectrolyte is present in an amount selected from the group consisting of from 0.5% to 99.999% by weight, preferably from 5.0% to 99.9% by weight, and more preferably from 10% to 99.5% by weight, based on the total combined dry weight of the indomethacin and at least one stabilizer, not including other excipients.

The composition of the invention and/or the indomethacin or pharmaceutically acceptable salts or cocrystals thereof in the composition of the invention is crystalline, amorphous, semi-crystalline, semi-amorphous, and co-crystal, and in mixtures thereof in any polymorph form.

For the preparation of the composition of the invention the following stabilizers mentioned as representative examples can be used: cellulose or derivatives thereof, polysaccharides, such as mannitol or sorbitol, polyvinylpyrrolidone, sodium lauryl sulfate, gelatin, cetostearyl alcohol, polyethylene glycols, acetic acid, polyvinylpyrrolidone-vinyl acetate copolymers, sodium dodecyl benzene sulfonate, sodium dodecyl-benzoyl sulfonate, tocopheryl polyethylene glycol succinate, urea, citric acid, sodium acetate, polyoxyethylene stearate, polyvinyl alcohol (PVA), polymethacrylate based polymers and copolymers, 4-(1,1,3,3-tetramethylbuthyl)-phenol polymer with ethylene oxide and formaldehyde (eg.: tyloxapol, Superione and triton), poloxamers (eg.: Pluronics, which are block copolymers of propylene oxide and ethylene oxide), poloxamines (eg., Tetronic, which is a tetrafunctional block copolymer), polycaprolactam-polyvinyl acetate-polyethylene glycol graft copolymer (Soluplus), D-alpha-tocopheryl polyethylene glycol succinate, poly(2-ethyl-2-oxazoline), poly(methyl vinyl ether), random copolymers of vinyl pyrrolidone and vinyl acetate, such as Plasdone S630.

Examples of useful ionic stabilizers include, but are not limited to polymers, biopolymers, polysaccharides, celluloses, alginates, phospholipids, and other nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide (PMMTMABr), hexyldesyltrimethylammonium bromide (HDMAB), and poly(vinylpyrrolidone)-2-dimethylaminoethyl methacrylate dimethyl sulfate.

Advantages of the composition of the invention include, but are not limited to: (1) smaller tablet or other solid dosage form size and beneficial transdermal/topical application; (2) lower doses of drug required to obtain the same pharmacological effect as compared to conventional forms of indomethacin; (3) increased bioavailability as compared to conventional forms of indomethacin; (4) improved pharmacokinetic profiles; (5) an increased rate of dissolution for indomethacin nanoparticles as compared to conventional forms of the same active compound; (6) modified metabolism of indomethacin nanoparticles.

Another aspect of the invention is a process for the preparation of nanoparticles according to which indomethacin or its pharmaceutically acceptable salt or co-crystal dissolved in an appropriate solution is mixed with a solution of one or more stabilizers and/or polyelectrolytes or a mixture thereof, if desired in the presence of a pharmaceutically acceptable acid or base in a flow reactor to obtain the nanostructured particles.

Preferably the process for the preparation of the composition of the invention comprises the steps of

(1) dissolving indomethacin or its pharmaceutically acceptable salt in a suitable solvent, if desired in the presence of one or more stabilizer or a mixture thereof;

(2) adding a solution comprising one or more polyelectrolytes and/or stabilizers or a mixture thereof, if desired in the presence of a pharmaceutically acceptable acid or base to the solution of step (1); and

(3) precipitating the nanoparticles from step (2).

Preferably the process for the preparation of the composition of the invention comprises the steps of

(1) dissolving indomethacin or its pharmaceutically acceptable salt in a suitable solvent in the presence of one or more stabilizer or a mixture thereof;

(2) adding a solution comprising one or more polyelectrolytes and/or stabilizers or a mixture thereof, if desired in the presence of a pharmaceutically acceptable acid or base to the solution of step (1); and

(3) precipitating the nanoparticles from step (2).

Preferably the process for the preparation of the composition of the invention comprises of the steps of

(1) dissolving indomethacin or its pharmaceutically acceptable salt in a suitable solvent in the presence of one or more stabilizer;

(2) adding a solution of pharmaceutically acceptable acid or base to the solution of step (1); and

(3) precipitating the nanoparticles from step (2).

The process of the invention is carried out by (a) using two different solvents miscible with each other, wherein indomethacin or its pharmaceutically acceptable salt is soluble only in one of them, or (b) using the same solvent in the two steps, where the polyelectrolyte complex of indomethacin or its pharmaceutically acceptable salt or co-crystal forms nanostructured complex particles, with the restriction that the applied polyelectrolyte and/or stabilizer(s) is/are soluble in the solvents used.

The microfluidics based flow reactor is described in the publication Microfluid Nanofluid DOI 10.1007/s 10404-008-0257-9 by I. Hornyak, B. Borcsek and F. Darvas.

If in the process of the invention two different solvents are used for the chemical precipitation then they are miscible with each other, where indomethacin is soluble only in one of them. Such solvents may preferably be dimethyl-sulfoxyde, ethanol, i-propanol, methanol and acetone.

The particle size of the nanoparticles made of indomethacin or its pharmaceutically acceptable salt or co-crystal may be influenced by the solvents used, the flow rate and the indomethacin:stabilizer ratio.

Another aspect of the invention is directed to the good/instantaneous redispersibility of the composition in biologically relevant mediums, e.g.; physiological saline solution, or pH=2.5 HCl solution.

Another aspect of the invention is a pharmaceutical formulation comprising the composition of the invention and other pharmaceutically acceptable auxiliary materials.

The pharmaceutical formulation of the invention can be formulated: (a) for administration selected from the group consisting of oral, pulmonary, rectal, colonic, intracisternal, intravaginal, intraperitoneal, ocular, otic, local, buccal, nasal, and topical administration; (b) into a dosage form selected from the group consisting of liquid, dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The formulation can be formulated by adding different types of excipients for oral administration in solid, liquid, vaginal, rectal, local (powders, ointments or drops), or topical administration, and the like.

A preferred dosage form of the formulation of the invention is a solid or liquid (cream/ointment) dosage form, although any pharmaceutically acceptable dosage form can be utilized.

For oral delivery nanoparticles can be also administered as their aqueous dispersion. This is a way of delivery without further processing after nanoparticle formation. However, poor stability of the drug or polymer in an aqueous environment or poor taste of the drug may require the incorporation of the colloidal particles into solid dosage forms, i.e. into capsules and tablets.

Alternatively, the aqueous dispersion can be incorporated into the solid dosage form as a liquid, for example by granulation of suitable fillers with the colloidal dispersion to form a granulation. Such granules can subsequently be filled into capsules or be compressed into tablets. Alternatively, through layering of the dispersion onto e.g. sugar-pellets a solid form for nanoparticles can be made. These ways of manufacturing is followed by a final coating step to obtain a film-coated tablet or film coated granules as the final dosage form.

The composition of the invention is suitable for parenteral (injection) application, which may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include, but not limited to water, ethanol, polyols (propyleneglycol, polyethylene-glycol, glycerol, and the like), suitable mixtures thereof, vegetable oils (such as olive oil) and injectable organic esters such as ethyl oleate. Maintenance of proper fluidity is essential in the use, for example, in the use of a coating such as lecithin, in the maintenance of the required particle size in the case of dispersions, and in the use of surfactants.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alginates, gelatin, poly(vinylpyrrolidone), sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolinite and bentonite; and j) lubricants, such as talc, calcium stearate, magnesium stearate, solid polyethylene glycols, sodium dodecyl sulfate, or mixtures thereof For capsules, tablets, and pills, the dosage forms may also comprise buffering agents.

Liquid dosage forms for oral administration include pharmaceutically acceptable emulsions, solutions, suspensions, syrups, and elixirs. Moreover, the liquid dosage form of indomethacin comprises inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters or mixtures of these substances, and the like.

Besides such inert diluents, the composition of the invention can also include adjuvants, such as wetting agents, emulsifying and suspending agents, sweetening, flavoring, and perfuming agents.

The compositions of the invention show enhanced lipophilicity/bioavailability/increased absorption and dissolution rate/reduced side effect, so they can be used in a decreased dosage as compared to conventional indomethacin form in the treatment of fever, inflammation, tense muscles, swellings and inflammation.

The novel nanoparticle containing indomethacin according to the present invention is also suitable for the treatment of fever, inflammation, tense muscles, swellings and inflammation.

A. Preferred Characteristics of the indomethacin nanoparticle composition of the invention

1. Increased Bioavailability

The compositions of the invention exhibit increased bioavailability, faster onset of action, reduced food effect and require smaller doses to achieve therapeutic effect as compared to prior known, conventional indomethacin formulations.

2. Dissolution Profiles of the Compositions of the Invention

The compositions containing indomethacin or pharmaceutically acceptable salts or cocrystals according to the invention are characterized by increased solubility due to the decreased particles size. Rapid dissolution of an administered active agent is preferable, as faster dissolution generally leads to faster onset of action and greater bioavailability.

3. Crystallographic Structure of Nanoparticles of the Invention

The chemical stability of solid drugs is affected by the crystalline state of said drug. Many drug substances exhibit polymorphism. Each crystalline state has different chemical reactivity. The stability of drugs in their amorphous form is generally lower than that of drugs in their crystalline form, because of the higher free-energy level of the amorphous state.

Change in crystalline state caused by mechanical stresses such as grinding may induce decreased chemical stability.

The chemical stability of solid drugs is also affected by the crystalline state of the drug through differences in surface area. For reaction that proceeds on the solid surface of drug, an increase in the surface area can increase the amount of drug participating in the reaction.

### EXAMPLE 1

Crystallographic Structure Determination

Stable amorphous/partly crystalline/crystalline/polymorph compositions containing indomethacin according to the invention shows significantly enhanced solubility due to its increased surface area when compared to a crystalline reference.

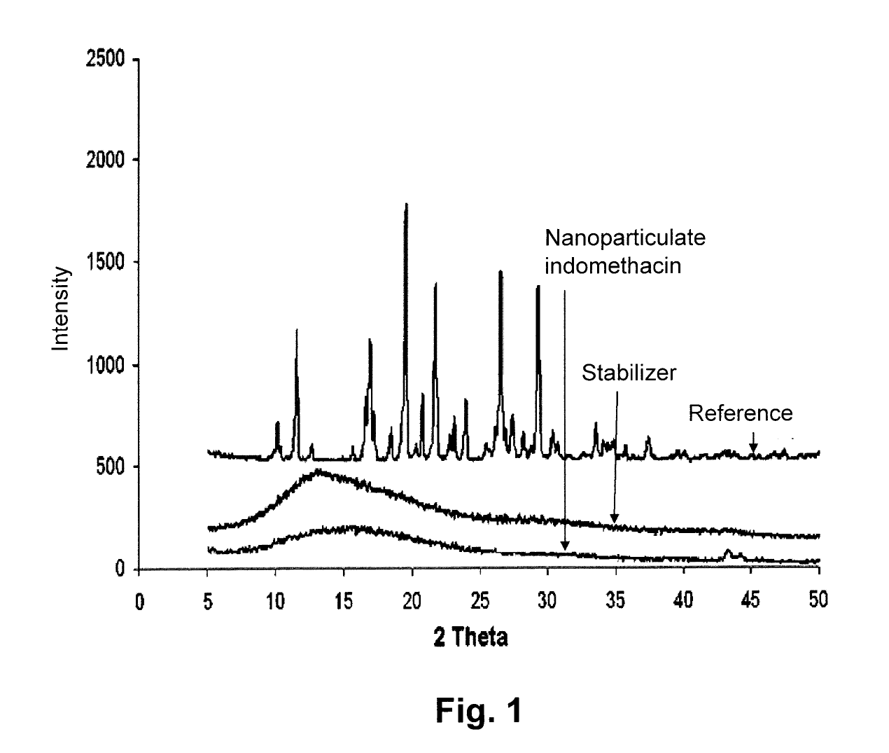

The structure of the indomethacin nanoparticles prepared by solvent-antisolvent precipitation method of example 4 was investigated by X-ray diffraction analysis (Philips PW1050/1870 RTG powder-diffractometer). The measurements showed that the nanoparticles are amorphous (see on FIG. 1).

FIG. 1: X-ray diffractograms of reference indomethacin and composition containing nanoparticulate indomethacin according to the invention

4. Redispersibility Profiles of the Composition of the Invention

An additional advantage of the composition of the present invention is that the dried nanoparticles stabilized by stabilizers or using traditional redispersants such as mannitol, sucrose can be redispersed instantaneously.

### EXAMPLE 2

Redispersibility Test

Redispersibility test was performed in distillate water to determine the solubility of the nanostructured composition containing indomethacin of example 4. 5 mg of freeze dried nanostructured composition containing indomethacin-Ca were redispersed in 1 mL distillate water under vigorous stirring. The particle size of the redispersed sample was determined by DLS method (Nanotrac instrument, Mictrotrac Co., USA).

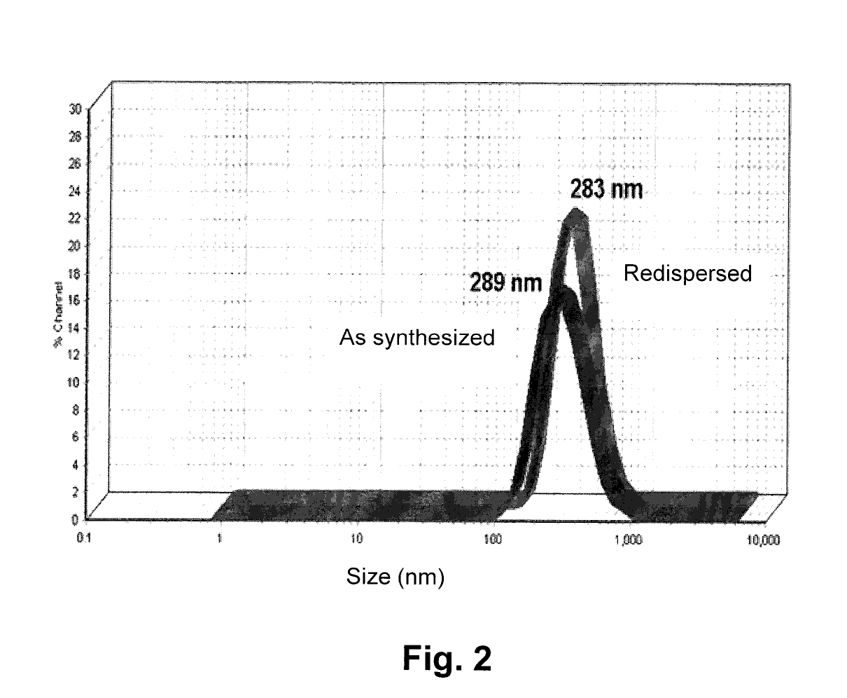

The characteristic particle size of the redispersed composition containing indomethacin (intensity-based average) is d=283 nm, while d(90) value is 425 nm, which is demonstrated in FIG. 2.

The significant benefit of the composition of the invention is that the indomethacin nanoparticle composition of the present invention can be redispersed after the drying/solid formulation procedure and the nanoparticles obtained by redispersion have similar average particle size to that of initial nanoparticles. Having the similar average particles size after the redispersion, the dosage form cannot lose the benefits afforded by the nanoparticle formation. A nano-size of the present invention is an average particle size of less than 500 nm.

FIG. 2: Size and size distribution of the indomethacin nanoparticles before and after the redispersion

5. Enhanced Lipophilicity of the Composition According to the Invention—Increased Absorption and Permeability

Due to the phospholipidic nature of cell membranes, a certain degree of lipophilicity is often a requirement for the drug compound, not only to be absorbed through the intestinal wall following oral administration but possibly also to exert its pharmacological action in the target tissue. (F. Kesisoglou et al./Advanced Drug Delivery Reviews 59 (2007) 631-644)

The lipophilicity of the indomethacin can be increased by using lipophilic stabilizers or/and stabilizers having lipophilic side groups and/or amphiphilic stabilizers during the precipitation-based preparation. Due to the lipophilic nature or lipophilic side groups of the stabilizer, not only the lipophilicity, but the absorption and the permeability of the indomethacin nanoparticles of the present invention can be increased.

For example using Chitosan as stabilizer, it can increase the paracellular permeability of intestinal epithelia which attributed to the transmucosal absorption enhancement.

Most amphiphilic copolymers employed for drug delivery purposes contain either a polyester or a poly(amino acid)-derivative as hydrophobic segment. Most of the polyesters of pharmaceutical interest belong to the poloxamer family, i.e. block-copolymers of polypropylene glycol and polyethylene glycol.

B. Compositions

The invention provides compositions containing indomethacin or pharmaceutically acceptable salts or co-crystals or mixture thereof which comprise at least one stabilizer to stabilize them sterically and/or electrostatically.

The stabilizers are preferably associated with the indomethacin or pharmaceutically acceptable salts or co-crystals thereof, but do not chemically react with the indomethacin or themselves.

The nanoparticles of the invention can be formed by complexation using biocompatible or biodegradable polyelectrolyte or can be prepared by solvent-antisolvent precipitation methods using stabilizer(s). The stability of the prepared colloid solution containing nanosized indomethacin particles can be increased by the addition of further stabilizer(s) which can act as a second steric or electrostatic stabilizer. Moreover, using further stabilizer the particle size of the composition containing indomethacin or pharmaceutically acceptable salts or co-crystals thereof according to the invention can be decreased and controlled.

6. Particle Size of Nanoparticles Formed by Indomethacin or Pharmaceutically Acceptable Salts, Co-Crystals or Mixtures Thereof According to the Invention

Nanoparticles according to the invention have an average particle size of less than 500 nm as measured by dynamic light scattering method.

By “an average particle size of less than 500 nm” it is meant that at least 50% of the nanoparticles containing indomethacin or pharmaceutically acceptable salts or co-crystals thereof have a particle size of less than the average, by number/intensity, i.e., less than about 500 nm, etc., when measured by the above-noted technique.

### EXAMPLE 3

Preparation of Nanostructured Composition Containing Indomethacin

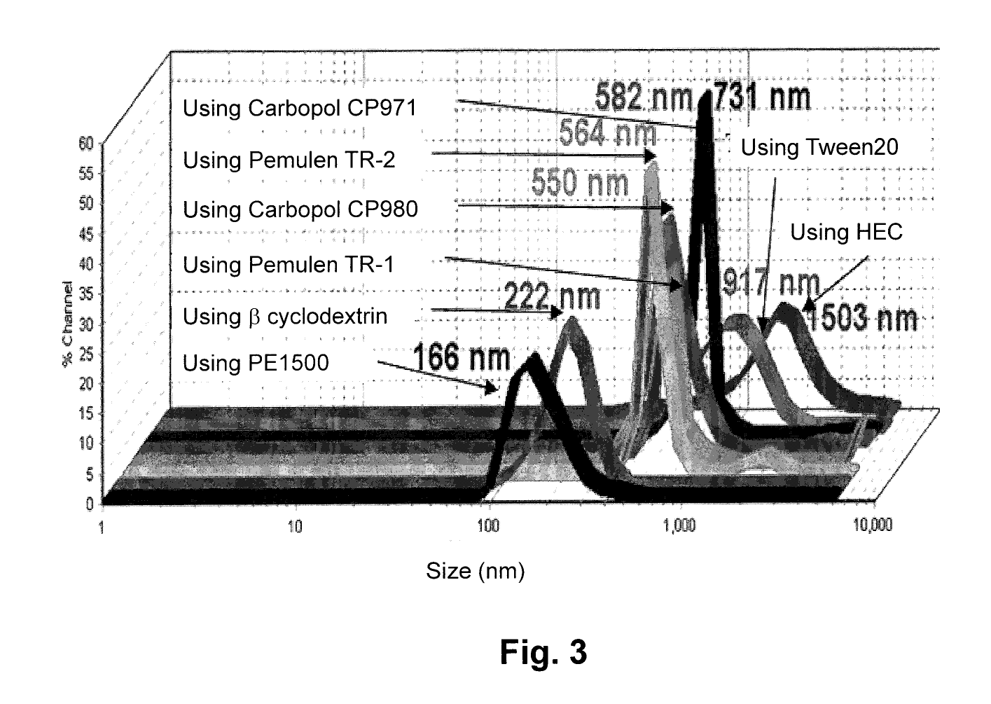

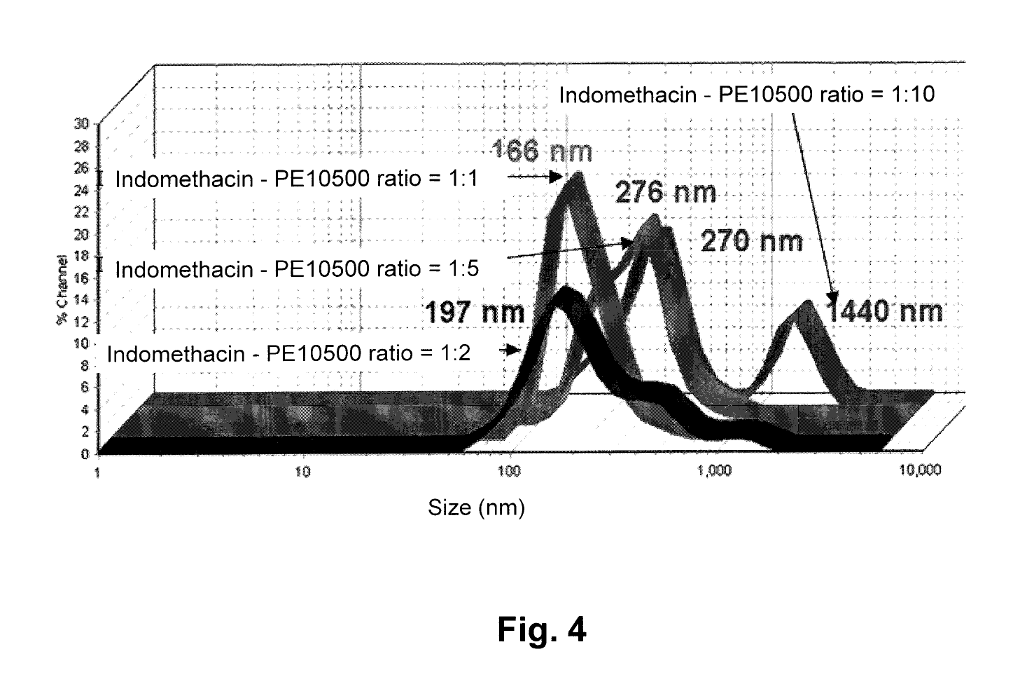

During the experiment nanoparticles containing indomethacin were prepared in a microfluidic based flow reactor. As a starting solution, 200 mg indomethacin (IMC) and 1 g Pluronic PE10500 were dissolved in a mixture of 90 mL DMSO and 10 ml distilled water. The prepared solution was passed into the first reactor unit with 1 mL/min flow rate using a first feeding unit. Meanwhile, using a second feeding unit, distilled water was passed into a mixing unit with 1 mL/min flow rate, where it was mixed with the solution containing indomethacin coming from the first reactor unit. The nanoparticles are continuously produced at atmospheric pressure due to the chemical precipitating effect of the distilled water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates, the pressure and the stabilizer (see FIG. 3). The size of the nanoparticles containing indomethacin can be controlled by the amount of the stabilizer (see FIG. 4).

FIG. 3: Size and size distribution of nanoparticles containing indomethacin using different stabilizers

FIG. 4: Size and size distribution of nanoparticles containing indomethacin using different indomethacin: stabilizer ratio

### EXAMPLE 4

Preparation of Nanostructured Composition Containing Indomethacin

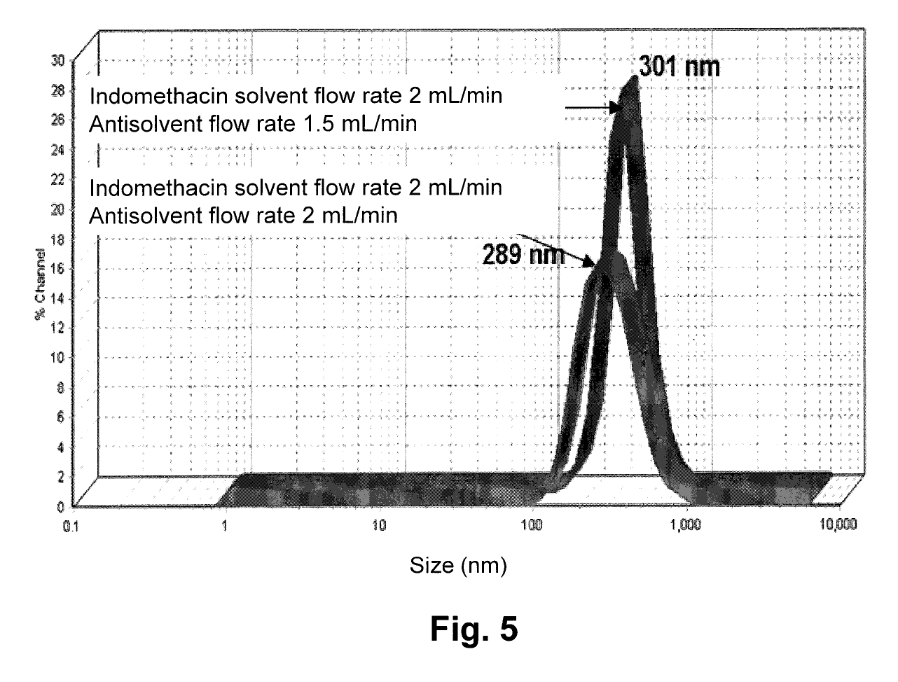

During the experiment nanoparticles containing indomethacin were prepared in a microfluidic based flow reactor. As a starting solution, 200 mg indomethacin (IMC) and 650 mg Eudragit RS100 were dissolved in 100 ml ethanol. The prepared solution was passed into the first reactor unit with 2 mL/min flow rate using a first feeding unit. Meanwhile, using a second feeding unit, sodium dodecyl sulphate solution of 0.05% concentration made with distilled water was passed into a mixing unit with 2 mL/min flow rate, where it was mixed with the solution containing indomethacin coming from the first reactor unit. The nanoparticles are continuously produced at atmospheric pressure due to the chemical precipitating effect of the distilled water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates, the pressure and the stabilizer (see FIG. 5). The particle size of the nanoparticles containing indomethacin was 289 nm in the best case (see Table 1).

FIG. 5: Size and size distribution of nanoparticles containing indomethacin using different antisolvent flow rates

Table 1: Effect of the flow rates on the particle size of the nanoparticles containing indomethacin

TABLE 1

| | Sample 1 | Sample 2 | Sample 3 | Sample 4 |
|---|---|---|---|---|
| API flow rate | 2 | 2 | 2 | 2 |
| (ml/min) | | | | |
| Antisolvent flow | 1 | 1.5 | 2 | 2.5 |
| rates (ml/min) | | | | |
| Particle size (DLS) | No | 301 | 289 | aggregation |
| (nm) | precipitation | | | |

### EXAMPLE 5

Formulation of Cream Comprising the Nanoparticles Containing Indomethacin

2 g Carbopol 980 was dissolved under vigorous stirring at room temperature in the aqueous colloidal solution comprising the nanoparticles containing indomethacin to obtain 100 ml of aqueous gel comprising the nanoparticles containing indomethacin as synthesized by the method described in example 4.

1. A stable nanostructured indomethacin composition comprising
(a) indomethacin or pharmaceutically acceptable salts, cocrystals or mixtures thereof; and
(b) at least one polyelectrolyte and/or stabilizer or mixtures thereof for stabilization of the nanoparticles; and optionally further stabilizers for steric and electrostatic stabilization;
wherein the nanostructured particles have a particle size of 500 nm or less;
and wherein the composition is prepared by controlled nano-precipitation in a flow reactor, preferably in a microfluidic based flow reactor; and preferably comprises stabilizers;
and wherein the stabilizer is selected from cellulose or derivatives thereof, polysaccharides, such as mannitol or sorbitol, polyvinylpyrrolidone, gelatin, cetostearyl alcohol, polyethylene glycols, acetic acid, polyvinylpyrrolidone-vinyl acetate copolymers, sodium dodecyl benzene sulfonate, sodium dodecyl-benzoyl sulfonate, tocopheryl polyethylene glycol succinate, urea, citric acid, sodium acetate, polyoxyethylene stearate, polyvinyl alcohol (PVA), polymethacrylate based polymers and copolymers, 4-(1,1,3,3-tetramethylbuthyl)-phenol polymer with ethylene oxide and formaldehyde groups (eg. tyloxapol, Superione and triton), poloxamers (eg. Pluronics, which are block copolymers of propylene oxide and ethylene oxide), poloxamines (eg. Tetronic, which is a tetrafunctional block copolymer), polyethylene glycol-polycaprolactam-polyvinyl acetate graft copolymer (Soluplus), D-alpha-tocopheryl polyethylene glycol succinate, poly(2-ethyl-2-oxazoline), poly(methyl vinyl ether), random copolymers of vinyl pyrrolidone and vinyl acetate, such as Plasdone S630, and mixtures thereof.

2. The composition according to claim 1 characterized in that the stabilizer is Pluronic PE 10500.

3. (canceled)

4. The composition according to claim 1 characterized in that
a) the particles have an average particle size of less than 500 nm; and/or
b) the composition has an increased solubility in water and/or in biologically relevant mediums as compared to conventional forms of indomethacin; and/or
c) the indomethacin is crystalline, amorphous, semi-crystalline, semi-amorphous phase, or in co-crystal form, or in mixtures thereof, in any polymorph form; and/or
d) the solid composition containing indomethacin is wetted and redispersed instantaneously in water and/or in biologically relevant mediums; and/or
e) the filtrate of the redispersed nanoparticles containing indomethacin is transparent and stable over time in water and/or in biologically relevant mediums; and/or
f) the composition is bioequivalent or characterized by improved pharmacokinetic profile as compared to reference or marketed API; and/or
g) tₘₐₓ is shorter or bioequivalent as compared to reference or marketed API; and/or
h) Cₘₐₓ is higher or bioequivalent as compared to reference or marketed API.

5. A process for the preparation of the composition according to claim 1 comprising precipitating the nanoparticles by adding a precipitating agent to the solution of indomethacin or pharmaceutically acceptable salts thereof made with a suitable solvent in a flow reactor, preferably a microfluidic based flow reactor, wherein the solution comprises one or more stabilizer according to claim 1, and the precipitating agent is water and optionally comprises one or more stabilizer.

6. The process according to claim 5 comprising the steps of
(1) dissolving indomethacin or its pharmaceutically acceptable salt in a suitable solvent, in the presence of one or more of the stabilizers according to claim 3;
(2) during the formulation step adding the solution of step (1) to another solution in the presence of a pharmaceutically acceptable acid or base; and
(3) forming the nanoparticles in step (2) by precipitating.

7. (canceled)

8. (canceled)

9. A method of treatment of fever, inflammation, tense muscles, swellings or inflammation by administering to an individual in need thereof an effective amount of the composition according to claim 1.

10. A pharmaceutical formulation comprising the composition according to claim 1 and other pharmaceutically acceptable auxiliary materials.
